(19) United States
(12) Patent Application Publication
(10) Pub. No.: US 2004/0109893 A1
(43) Pub. Date: Jun. 10, 2004

(54) Sustained release dosage forms of anesthetics for pain management

(76) Inventors: Guohua Chen, Sunnyvale, CA (US); David T. Priebe, Seattle, WA (US); Roy Bannister, Hollister, CA (US); Paul Houston, Hayward, CA (US); Lothar Walter Kleiner, Los Altos, CT (US)

Correspondence Address: WOODCOCK WASHBURN LLP, ONE LIBERTY PLACE, 46TH FLOOR, 1650 MARKET STREET, PHILADELPHIA, PA, 19103, US

(21) Appl. No.: 10/699,521

(22) Filed: Oct. 31, 2003

Related U.S. Application Data
(63) Continuation-in-part of application No. 10/606,969, filed on Jun. 25, 2003.
(60) Provisional application No. 60/391,867, filed on Jun. 25, 2002.

(51) Int. Cl. A61K 9/22

(52) U.S. Cl. 424/468

(57) ABSTRACT

Drug delivery systems and kits are provided that release an anesthetic, such as bupivacaine, over a short duration. Methods of administering and preparing such systems are also provided. Drug delivery systems include a short duration gel vehicle and an anesthetic dissolved or dispersed in the gel vehicle. The gel vehicle comprises a low molecular weight bioerodible, biocompatible polymer and a water-immiscible solvent in an amount effective to plasticize the polymer and form a gel with the polymer. In some instances, a component solvent is used along with the water-immiscible solvent. An efficacy ratio, which is one way to measure the efficacy of a delivery system, can be controlled based on, for example, the construction of the gel vehicle to achieve a desired release profile.

This application is a continuation-in-part of U.S. patent application Ser. No. 10/606,969, filed Jun. 25, 2003, incorporated herein by reference, which claims the benefits of U.S. Provisional Application No. 60/391,867, filed on Jun. 25, 2002, incorporated herein by reference.

### FIELD OF THE INVENTION

The present invention relates to sustained release dosage forms and kits comprising an anesthetic which can be applied to a desired location. The present invention also relates to methods of preparing and administering the dosage forms.

### BACKGROUND OF THE INVENTION

Management of pain, for example post-surgical pain, is an important step for the road to recovery for a patient. Although many factors influence what pain relief therapy is optimal for each patient, therapies that are easily administered are strongly desired.

One therapy for management of post-operative pain is to use local anesthetics, e.g. bupivacaine. Bupivacaine is a long acting, local anesthetic administrated by local infiltration for peripheral nerve block and caudal and lumbar epidural block. As widely understood in the art, bupivacaine hydrochloride is available for treating post-operative pain, for example, as a parenteral solution alone, a solution in dextrose injection, and a combination with epinephrine.

Post-operative pain that accompanies all types of procedures, such as major surgeries (e.g., thoracotomy, aortic repair, and bowel resection), intermediate surgeries (e.g., cesarean section, hysterectomy, and appendectomy), and minor surgeries (e.g., hemiorrhaphy, laparoscopy, arthroscopy, breast biopsy) can be debilitating and may require pain treatment for three to five days after surgery. A local anesthetic solution such as 0.5% bupivacaine hydrochloride with epinephrine, however, provides local analgesia for only about four to nine hours. As a result, standard post-operative therapies using anesthetics such as bupivacaine requires either frequent injection or constant intravenous infusion.

There remains a great need for drug delivery systems comprising anesthetics which can provide sustained release over a short duration. A need also exists for single administration anesthetic delivery systems which provide sustained release over several days.

### SUMMARY OF THE INVENTION

Drug delivery systems and kits that release an anesthetic, such as bupivacaine, over a short duration are provided by the present invention. Methods of administering and preparing such systems are also provided. Drug delivery systems, for example sustained release dosage forms, in accordance with the present invention include a short duration gel vehicle and an anesthetic dissolved or dispersed in the gel vehicle. The gel vehicle comprises a low molecular weight bioerodible, biocompatible polymer and a water-immiscible solvent in an amount effective to plasticize the polymer and form a gel with the polymer. In some instances, a component solvent is used along with the water-immiscible solvent.

Dosage forms of the present invention represent an advantage over conventional systemic pain treatments which may require either frequent injections or constant infusion of an intravenous solution. With respect to post-operative analgesic therapy, for example, drug delivery systems of the present invention that can release a fixed amount of anesthetic, such as bupivacaine, to a surgical site for an extended period of time is advantageous over the standard systemic post-operative analgesic therapy of frequent injections or constant intravenous infusion. Advantages are also achieved when dosage forms of the present application are administered only once. On the other hand, it is also contemplated that dosage forms of the invention can be administered with repeated dosages.

A purpose of this invention is to develop short duration sustained release dosage forms of anesthetics, for example bupivacaine, that can be applied to subjects for managing pain, e.g., post-operative pain. An efficacy ratio, which is one way to measure of the efficacy of a delivery system, is the ratio between a maximum achieved concentration of beneficial agent (Cₘₐₓ), e.g. an anesthetic, achieved shortly after administration of the dosage form, and an average concentration of the beneficial agent measured over a given length of time after the maximum concentration occurs (C_{average}), for example between days 2 and 9 after administration. The efficacy ratio can be controlled based on, for example, the construction of the gel vehicle to achieve a desired release profile. The ratio of polymer and solvent in the gel vehicle can affect the efficacy ratio, as can the choice of a water-immiscible solvent or solvent mixtures, a component solvent and/or the choice of an excipient. In addition, molecular weight of the polymer and/or the average particle size of the beneficial agent can also impact the efficacy ratio. Efficacy ratios can be tailored based on the needs of the subject as well as the beneficial agent being administered and may range from approximately 1 to approximately 200. In some instances, efficacy ratios may range from about 5 to about 100.

For post-surgical pain management, it is usually desired to deliver a drug to achieve a sufficiently high Cₘₐₓ of the anesthetic agent to control the pain almost immediately and then maintain a sustained level of anesthetic over a certain duration. In this instance, a higher efficacy ratio may be desirable. In other situations, however, to reduce potential side effects from a high dosage of the drug, it may be useful to maintain a tightly controlled level of active agent either in systemic circulation or distribution in the local tissues. For this type of situation, a lower efficacy ratio may be desirable. As such, because of varying patient and therapy needs, it is desirable to control the efficacy ratio of a drug delivery dosage form.

With respect to the ratio between the polymer and the solvent embodied by the present invention, ratios of between about 5:95 and about 90:10, between about 20:80 and about 80:20, and/or between about 30:70 and about 75:25 are contemplated.

Short duration sustained release dosage forms, for example injectable depot gel compositions as discussed by co-pending U.S. patent application Ser. No. 10/606,969 incorporated herein by reference, can provide both systemic and local delivery of a beneficial agent to a subject over a short duration of time. In particular, short duration sustained release dosage forms can release the beneficial agent, e.g. an anesthetic such as bupivacaine, to the subject being treated over a period of less than or equal to about two weeks after administration. Other embodiments of the present invention control the release over a period of less than or equal to about seven days. Still other embodiments can control release of the beneficial agent in a period of between about 24 hours and seven days.

Although there is no limit to the anesthetics that are suitable for use in the present invention, U.S. Pat. No. 6,432,986 incorporated herein by reference provides several examples, in one aspect of the present invention, the anesthetic is selected from the group consisting of: bupivacaine, levo-bupivacaine, ropivacaine, levo-ropivacaine, tetracaine, etidocaine, levo-etidocaine, dextro-etidocaine, levo-etidocaine, dextro-etidocaine, levo-mepivacaine, and combinations thereof. In other aspects, the anesthetic comprises bupivacaine.

In additional aspects of the present invention, the solvents of the gel vehicle have a miscibility in water of less than or equal to about 7 weight % at 25° C. It is also an embodiment of the present invention that the dosage form is free of solvents having a miscibility in water that is greater than 7 weight % at 25° C. Although many solvents are suitable for the present invention, in one aspect of the present invention, the solvent is selected from the group consisting of: an aromatic alcohol, lower alkyl esters of aryl acids, lower aralkyl esters of aryl acids; aryl ketones, aralkyl ketones, lower alkyl ketones, lower alkyl esters of citric acid, and combinations thereof. Useful solvents used in the present invention include, but are not limited to, benzyl alcohol, benzyl benzoate, ethyl benzoate, triacetin, and mixtures thereof.

Further aspects of the present invention include sustained release dosage forms as discussed above further comprising a component solvent selected from the group consisting of: triacetin, diacetin, tributyrin, triethyl citrate, tributyl citrate, acetyl triethyl citrate, acetyl tributyl citrate, triethylglycerides, triethyl phosphate, diethyl phthalate, diethyl tartrate, mineral oil, polybutene, silicone fluid, glylcerin, ethylene glycol, polyethylene glycol, octanol, ethyl lactate, propylene glycol, propylene carbonate, ethylene carbonate, butyrolactone, ethylene oxide, propylene oxide, N-methyl-2-pyrrolidone, 2-pyrrolidone, glycerol formal, methyl acetate, ethyl acetate, methyl ethyl ketone, dimethylformamide, dimethyl sulfoxide, tetrahydrofuran, caprolactam, decylmethylsulfoxide, oleic acid, and 1-dodecylazacyclo-heptan-2-one, and combinations thereof.

In other embodiments of the present invention, the gel vehicle comprises a lactic acid-based polymer or a copolymer of lactic acid and glycolic acid (PLGA). Other embodiments use caprolactone-based polymers. Polymers can also be selected from the group consisting of: polylactides, polyglycolides, poly(caprolactone), polyanhydrides, polyamines, polyesteramides, polyorthoesters, polydioxanones, polyacetals, polyketals, polycarbonates, polyphosphoesters, polyesters, polybutylene terephthalate, polyorthocarbonates, polyphosphazenes, succinates, poly(malic acid), poly(amino acids), polyvinylpyrrolidone, polyethylene glycol, polyhydroxycellulose, polysaccharides, chitin, chitosan, hyaluronic acid, and copolymers, terpolymers and mixtures thereof. Polymers used in the present invention can comprise an ester end group or a carboxylic acid end group. Furthermore, polymers can have weight average molecular weights of between about 1,000 and about 10,000, between about 3,000 and about 10,000, between about 3,000 and about 8,000, between about 4,000 and about 6,000, and/or about 5,000.

Dosage forms in accordance with the present invention comprise from about 0.1% to about 50% anesthetic by weight, about 0.5% to about 40% anesthetic by weight, and/or about 1% to about 30% anesthetic by weight.

Other aspects of the present invention include anesthetic particles having an average particle size of less than about 250 μm, between about 5 μm and 250 μm, between about 20 μm and about 125 μm, and/or between about 38 μm and about 63 μm.

Still additional aspects in accordance with the present invention include sustained release dosage forms as discussed above comprising at least one of the following: an excipient, such as stearic acid, an emulsifying agent, a pore former, a solubility modulator for the anesthetic, and an osmotic agent.

Another embodiment of the invention includes sustained release dosage forms of an anesthetic comprising a short duration gel vehicle comprising a low molecular weight lactic acid-based polymer and a water-immiscible solvent, in an amount effective to plasticize the polymer and form a gel therewith; an anesthetic comprising bupivacaine, wherein the anesthetic is dissolved or dispersed in the gel vehicle; and a controllable efficacy ratio to achieve a release profile; wherein the weight average molecular weight of the lactic acid-based polymer is between about 3,000 and about 10,000.

An additional embodiment of the invention includes sustained release dosage forms of an anesthetic comprising a short duration gel vehicle comprising a low molecular weight copolymer of lactic acid and glycolic acid (PLGA) and a water-immiscible solvent, in an amount effective to plasticize the polymer and form a gel therewith; an anesthetic comprising bupivacaine, wherein the anesthetic is dissolved or dispersed in the gel vehicle; and a controllable efficacy ratio to achieve a release profile; wherein the weight average molecular weight of the co polymer is between about 3,000 and about 10,000.

The invention also includes methods of treating local pain of a subject using a sustained release dosage form, the methods comprising: administering a short duration sustained release dosage form comprising a gel vehicle, which comprises a low molecular weight bioerodible, biocompatible polymer, and a water-immiscible solvent in an amount effective to plasticize the polymer and form a gel therewith; and an anesthetic dissolved or dispersed in the gel vehicle.

Other methods include treating post-surgical local pain of a subject using a sustained release dosage form, the methods comprising: administering once a short duration sustained release dosage form comprising a gel vehicle, which comprises a low molecular weight bioerodible, biocompatible lactic acid-based polymer or copolymer of lactic acid and glycolic acid (PLGA), and a water-immiscible solvent in an amount effective to plasticize the polymer and form a gel therewith; an anesthetic comprising bupivacaine dissolved or dispersed in the gel vehicle; and a controllable efficacy ratio to achieve a release profile.

The dosage forms of the invention can be once administered or repeatedly administered. The dosage forms can be applied topically to the local pain. In other aspects of the invention, the dosage form is injected at a location near the local pain. The anesthetic can be delivered systemically or locally. Delivery of the anesthetic can also be to multiple sites, for example, at multiple locations surrounding the local pain.

Another aspect of the invention includes methods of preparing a sustained release dosage form, the method comprising: preparing a short duration gel vehicle comprising a low molecular weight bioerodible, biocompatible polymer and a water-immiscible solvent in an amount effective to plasticize the polymer and form a gel therewith to create a polymer/solvent solution or gel; equilibrating the polymer/solvent solution or gel until a clear homogeneous solution or gel is achieved, at for example, a temperature range of room temperature to 65° C.; dissolving or dispersing an anesthetic into the polymer/solvent solution or gel; blending the anesthetic and the polymer/solvent solution or gel to form a sustained release dosage form; and controlling an efficacy ratio to achieve a release profile.

Also in accordance with the present invention, kits are provided for the administration of a sustained delivery of an anesthetic to local pain of a subject comprising: a short duration gel vehicle comprising a low molecular weight bioerodible, biocompatible polymer and a water-immiscible solvent, in an amount effective to plasticize the polymer and form a gel therewith; an anesthetic dissolved or dispersed in the gel vehicle; and optionally, one or more of the following: an excipient, such as stearic acid, an emulsifying agent, a pore former, a solubility modulator for the anesthetic, optionally associated with the anesthetic, and an osmotic agent; wherein at the least anesthetic, optionally associated with the solubility modulator, is maintained separated from the solvent until the time of administration of the anesthetic to the subject.

These and other embodiments will readily occur to those or ordinary skill in the art in view of the disclosure herein.

### BRIEF DESCRIPTION OF THE DRAWINGS

FIG. 1 is a graph illustrating the in vivo release profile of bupivacaine hydrochloride obtained from depot formulations of the present invention (formulations 1-2).

FIG. 2 is a graph illustrating the in vivo release profile of bupivacaine base obtained from depot formulations of the present invention (formulations 3-4).

FIG. 3 is a graph illustrating the early part of in vivo release profile (up to day 7) of bupivacaine base obtained from a depot formulation of the present invention (formulation 4).

FIG. 4 is a graph illustrating the in vivo release profile of bupivacaine obtained from depot formulations of the present invention (formulations 5-6).

FIG. 5 is a graph illustrating the in vivo release profile of bupivacaine obtained from depot formulations of the present invention (formulations 6-7).

FIG. 6 is a graph illustrating the in vivo release profile of bupivacaine obtained from depot formulations of the present invention (formulations 7-8).

FIG. 7 is a graph illustrating the in vivo release profile of bupivacaine obtained from depot formulations of the present invention (formulations 8-9).

FIG. 8 is a graph illustrating the in vivo release profile of bupivacaine obtained from depot formulations of the present invention (formulations 10-11).

FIG. 9 is a graph illustrating the in vivo release profile of bupivacaine obtained from depot formulations of the present invention (formulations 10, 12).

FIG. 10 is a graph illustrating the early part of in vivo release profile (up to day 4) of bupivacaine obtained from depot formulations of the present invention (formulations 10, 12).

FIG. 11 is a graph illustrating the in vivo release profile of bupivacaine obtained from depot formulations of the present invention (formulations 12, 13).

FIG. 12 is a graph illustrating the early part of in vivo release profile (up to day 4) of bupivacaine obtained from depot formulations of the present invention (formulations 12, 13).

FIG. 13 is a DSC diagram of the low molecular weight PLGA with an ester end group used to make various formulations of the present invention (formulations 2, 4, 5, 6, and 7).

FIG. 14 is a DSC diagram of the low molecular weight PLGA with a carboxyl end group used to make a various formulations of the present invention (formulations 8 and 13).

FIG. 15 is a graph illustrating the in vitro degradation profile of PLGA polymers of varying molecular weights with different end groups.

### DETAILED DESCRIPTION

The present invention is directed to drug delivery systems and kits that release an anesthetic, such as bupivacaine, over a short duration. Methods of administering and preparing such systems are also provided. Drug delivery systems in accordance with the present invention include a short duration gel vehicle and an anesthetic dissolved or dispersed in the gel vehicle. The gel vehicle comprises a low molecular weight bioerodible, biocompatible polymer and a water-immiscible solvent in an amount effective to plasticize the polymer and form a gel with the polymer. In some instances, a component solvent is used along with the water-immiscible solvent. An efficacy ratio, which is one way to measure of the efficacy of a delivery system, can be controlled based on, for example, the construction of the gel vehicle to achieve a desired release profile. The ratio of polymer and solvent in the gel vehicle can affect the efficacy ratio, as can the choice of a water-immiscible solvent or solvent mixtures, a component solvent and/or the choice of an excipient. In addition, molecular weight of the polymer and/or the average particle size of the beneficial agent can also impact the efficacy ratio. Efficacy ratios can be tailored based on the needs of the subject as well as the beneficial agent being administered and may range from approximately 1 to approximately 200. In some instances, efficacy ratios may range from about 5 to about 100.

For post-surgical pain management, it is usually desired to deliver a drug to achieve a sufficiently high Cₘₐₓ of the anesthetic agent to control the pain almost immediately and then maintain a sustained level of anesthetic over a certain duration. In this instance, a higher efficacy ratio may be desirable. In other situations, however, to reduce potential side effects from a high dosage of the drug, it may be useful to maintain a tightly controlled level of active agent either in systemic circulation or distribution in the local tissues. For this type of situation, a lower efficacy ratio may be desirable. As such, because of varying patient and therapy needs, it is desirable to control the efficacy ratio of a drug delivery dosage form.

Generally, the compositions of the invention are gel-like and form with a substantially homogeneous non-porous structure throughout the implant upon implantation and during drug delivery, even as it hardens. Furthermore, while the polymer gel implant will slowly harden when subjected to an aqueous environment, the hardened implant may maintain a rubbery (non-rigid) composition with the glass transition temperature T_{g} being below 37° C.

When the composition is intended for implantation by injection, the viscosity optionally may be modified by emulsifiers and/or thixotropic agents to obtain a gel composition having a viscosity low enough to permit passage of the gel composition through a needle. Also, pore formers and solubility modulators of the beneficial agent may be added to the implant systems to provide desired release profiles from the implant systems, along with typical pharmaceutical excipients and other additives that do not change the beneficial aspects of the present invention. The addition of a solubility modulator to the implant system may enable the use of a solvent having a solubility of 7% or greater in the implant system with minimal burst and sustained delivery under particular circumstances. However, it is presently preferred that the implant system utilize at least one solvent having a solubility in water of less than 7% by weight, whether the solvent is present alone or as part of a solvent mixture. It has also been discovered that when mixtures of solvents which include a solvent having 7% or less by weight solubility in water and one or more miscible solvents, optionally having greater solubility, are used, implant systems exhibiting limited water uptake and minimal burst and sustained delivery characteristics are obtained.

Definitions

In describing and claiming the present invention, the following terminology will be used in accordance with the definitions set out below.

The singular forms “a,” “an” and “the” include plural referents unless the context clearly dictates otherwise. Thus, for example, reference to “a solvent” includes a single solvent as well as a mixture of two or more different solvents, reference to “an anesthetic” includes a single anesthetic as well as two or more different anesthetics in combination, and the like.

The term “efficacy ratio” is defined as Cₘₐₓ/C_{averge}. Cₘₐₓ is a maximum achieved concentration of a beneficial agent, e.g. an anesthetic, achieved shortly after administration of the dosage form. C_{average} is an average concentration of the beneficial agent measured after the maximum concentration occurs for a given length of time based on the release duration of the dosage form. For example, for a dosage form with a seven day duration for release, Cₘₐₓ is measured at 1 hour and C_{averge} is measured over days 1 through 7.

The phrase “dissolved or dispersed” is intended to encompass all means of establishing a presence of beneficial agent in the gel composition and includes dissolution, dispersion, suspension and the like.

The term “systemic” means, with respect to delivery or administration of a beneficial agent to a subject, that the beneficial agent is detectable at a biologically-significant level in the blood plasma of the subject.

The term “local” means, with respect to delivery or administration of a beneficial agent to a subject, that the beneficial agent is delivered to a localized site in the subject but is not detectable at a biologically significant level in the blood plasma of the subject.

The terms “short period” or “short duration” are used interchangeably and refer to a period of time over which release of a beneficial agent from the depot gel composition of the invention occurs, which will generally be equal to or less than two weeks, preferably about 24 hours to about 2 weeks, preferably about 10 days or shorter; preferably about 7 days or shorter, more preferably about 3 days to about 7 days.

The term “gel vehicle” means the composition formed by mixture of the polymer and solvent in the absence of the beneficial agent.

The term “solubility modulator” means, with respect to the beneficial agent, an agent that will alter the solubility of the beneficial agent, with reference to polymer solvent or water, from the solubility of beneficial agent in the absence of the modulator. The modulator may enhance or retard the solubility of the beneficial agent in the solvent or water. However, in the case of beneficial agents that are highly water soluble, the solubility modulator will generally be an agent that will retard the solubility of the beneficial agent in water. The effects of solubility modulators of the beneficial agent may result from interaction of the solubility modulator with the solvent, or with the beneficial agent itself, such as by the formation of complexes, or with both. For the purposes hereof, when the solubility modulator is “associated” with the beneficial agent, all such interactions or formations as may occur are intended. Solubility modulators may be mixed with the beneficial agent prior to its combination with the viscous gel or may be added to the viscous gel prior to the addition of the beneficial agent, as appropriate.

The terms “subject” and “patient” mean, with respect to the administration of a composition of the invention, an animal or a human being.

Since all solvents, at least on a molecular level, will be soluble in water (i.e., miscible with water) to some very limited extent, the term “immiscible” as used herein means that 7% or less by weight, preferably 5% or less, of the solvent is soluble in or miscible with water. For the purposes of this disclosure, solubility values of solvent in water are considered to be determined at 25° C. Since it is generally recognized that solubility values as reported may not always be conducted at the same conditions, solubility limits recited herein as percent by weight miscible or soluble with water as part of a range or upper limit may not be absolute. For example, if the upper limit on solvent solubility in water is recited herein as “7% by weight,” and no further limitations on the solvent are provided, the solvent “triacetin,” which has a reported solubility in water of 7.17 grams in 100 ml of water, is considered to be included within the limit of 7%. A solubility limit in water of less than 7% by weight as used herein does not include the solvent triacetin or solvents having solubilities in water equal to or greater than triacetin.

The term “bioerodible” refers to a material that gradually decomposes, dissolves, hydrolyzes and/or erodes in situ. Generally, the “bioerodible” polymers herein are polymers that are hydrolyzable, and bioerode in situ primarily through hydrolysis.

The term “low molecular weight (LMW) polymer” refers to bioerodible polymers having a weight average molecular weight ranging from about 1,000 to about 10,000; preferably from about 3,000 to about 10,000; more preferably from about 3,000 to about 8,000, more preferably from about 4,000 to about 8,000; and more preferably the low molecular weight polymer has a molecular weight of about 7,000, about 6,000, about 5,000, about 4,000 and about 3,000 as determined by gel permeation chromatography (GPC).

The polymer, solvent and other agents of the invention must be “biocompatible”; that is they must not cause necrosis and have acceptable irritation or inflammation responses in the environment of use. The environment of use is a fluid environment and may comprise a subcutaneous, intramuscular, intravascular (high/low flow), intramyocardial, adventitial, intratumoral, or intracerebral portion, wound sites, tight joint spaces or body cavity of a human or animal.

The term “alkyl” as used herein refers to a saturated hydrocarbon group typically although not necessarily containing I to about 30 carbon atoms, such as methyl, ethyl, n-propyl, isopropyl, n-butyl, isobutyl, t-butyl, octyl, decyl, and the like, as well as cycloalkyl groups such as cyclopentyl, cyclohexyl and the like. Generally, although again not necessarily, alkyl groups herein contain 1 to about 12 carbon atoms. The term “lower alkyl” intends an alkyl group of 1 to 6 carbon atoms, preferably 1 to 4 carbon atoms. “Substituted alkyl” refers to alkyl substituted with one or more substituent groups, and the terms “heteroatom-containing alkyl” and “heteroalkyl” refer to alkyl in which at least one carbon atom is replaced with a heteroatom. If not otherwise indicated, the terms “alkyl” and “lower alkyl” include linear, branched, cyclic, unsubstituted, substituted, and/or heteroatom-containing alkyl or lower alkyl.

The term “aryl” as used herein, and unless otherwise specified, refers to an aromatic substituent containing a single aromatic ring or multiple aromatic rings that are fused together, linked covalently, or linked to a common group such as a methylene or ethylene moiety. Preferred aryl groups contain one aromatic ring or two fused or linked aromatic rings, e.g., phenyl, naphthyl, biphenyl, diphenylether, diphenylamine, benzophenone, and the like, and most preferred aryl groups are monocyclic. “Substituted aryl” refers to an aryl moiety substituted with one or more substituent groups, and the terms “heteroatom-containing aryl” and “heteroaryl” refer to aryl in which at least one carbon atom is replaced with a heteroatom. Unless otherwise indicated, the term “aryl” includes heteroaryl, substituted aryl, and substituted heteroaryl groups.

The term “aralkyl” refers to an alkyl group substituted with an aryl group, wherein alkyl and aryl are as defined above. The term “heteroaralkyl” refers to an alkyl group substituted with a heteroaryl group. Unless otherwise indicated, the term “aralkyl” includes heteroaralkyl and substituted aralkyl groups as well as unsubstituted aralkyl groups. Generally, the term “aralkyl” herein refers to an aryl-substituted lower alkyl group, preferably a phenyl substituted lower alkyl group such as benzyl, phenethyl, 1-phenylpropyl, 2-phenylpropyl, and the like.

I. Injectable Depot Compositions:

As described previously, injectable depot compositions for delivery of beneficial agents over a short duration of time may be formed as viscous gels prior to injection of the depot into a subject. The viscous gel supports dispersed beneficial agent to provide appropriate delivery profiles, which include those having controlled initial burst, of the beneficial agent as the beneficial agent is released from the depot over time.

The polymer, solvent and other agents of the invention must be biocompatible; that is they must not cause irritation or necrosis in the environment of use. The environment of use is a fluid environment and may comprise a subcutaneous, intramuscular, intravascular (high/low flow), intramyocardial, adventitial, intratumoral, or intracerebral portion, wound sites, tight joint spaces or body cavity of a human or animal. In certain embodiments, the beneficial agent may be administered locally to avoid or minimize systemic side effects. Gels of the present invention containing a beneficial agent may be injected/implanted directly into or applied as a coating to the desired location, e.g., subcutaneous, intramuscular, intravascular, intramyocardial, adventitial, intratumoral, or intracerebral portion, wound sites, tight joint spaces or body cavity of a human or animal.

Typically, the viscous gel will be injected from a standard hypodermic syringe, a catheter or a trocar, that has been pre-filled with the beneficial agent-viscous gel composition as the depot. It is often preferred that injections take place using the smallest size needle (i.e., smallest diameter) or catheter to reduce discomfort to the subject when the injection is in a subcutaneous, intramuscular, intravascular (high/low flow), intramyocardial, adventitial, intratumoral, or intracerebral portion, wound sites, tight joint spaces or body cavity of a human or animal. It is desirable to be able to inject gels through a needle or a catheter ranging from 16 gauge and higher, preferably 20 gauge and higher, more preferably 22 gauge and higher, even more preferably 24 gauge and higher. With highly viscous gels, i.e., gels having a viscosity of about 100 poise or greater, injection forces to dispense the gel from a syringe having a needle in the 20-30 gauge range may be so high as to make the injection difficult or reasonably impossible when done manually. At the same time, the high viscosity of the gel is desirable to maintain the integrity of the depot after injection and during the dispensing period and also facilitate desired suspension characteristics of the beneficial agent in the gel.

A composition of a polymer and polymer solvent that optionally includes an agent that imparts thixotropic characteristics to the viscous gel formed by the polymer solvent and polymer provides certain advantages. A thixotropic gel exhibits reduced viscosity when subjected to shear force. The extent of the reduction is in part a function of the shear rate of the gel when subjected to the shearing force. When the shearing force is removed, the viscosity of the thixotropic gel returns to a viscosity at or near that which it displayed prior to being subjected to the shearing force. Accordingly, a thixotropic gel may be subjected to a shearing force when injected from a syringe or a catheter, which temporarily reduces its viscosity during the injection process. When the injection process is completed, the shearing force is removed and the gel returns very near to its previous state.

Significant shear thinning properties of the injectable composition allow for a minimally invasive delivery, via a needle or a catheter, of a beneficial agent to various sites on an external and/or internal surface of the body. Further injection through the needle or injection catheter permits precise administration of a desirable amount of the composition at a desired location, with significant retention of the depot gel composition at the site of delivery while providing for sustained delivery of the beneficial agent from the site of administration. In certain embodiments, the injection catheter may include a metering device or an additional device to assist in the precise delivery of the composition.

The Bioerodible, Biocompatible Polymer:

Polymers that are useful in conjunction with the methods and compositions of the invention are bioerodible, i.e., they gradually degrade e.g., enzymatically or hydrolyze, dissolve, physically erode, or otherwise disintegrate within the aqueous fluids of a patient's body. Generally, the polymers bioerode as a result of hydrolysis or physical erosion, although the primary bioerosion process is typically hydrolysis or enzymatic degradation.

Such polymers include, but are not limited to polylactides, polyglycolides, polyanhydrides, polyamines, polyesteramides, polyorthoesters, polydioxanones, polyacetals, polyketals, polycarbonates, polyorthocarbonates, polyphosphazenes, succinates, poly(malic acid), poly(amino acids), polyvinylpyrrolidone, polyethylene glycol, polyhydroxycellulose, polyphosphoesters, chitin, chitosan, hylauronic acid and copolymers, terpolymers and mixtures thereof.

Presently preferred polymers are polylactides, that is, a lactic acid-based polymer that can be based solely on lactic acid or can be a copolymer based on lactic acid and glycolic acid which may include small amounts of other comonomers that do not substantially affect the advantageous results which can be achieved in accordance with the present invention. As used herein, the term “lactic acid” includes the isomers L-lactic acid, D-lactic acid, DL-lactic acid and lactide while the term “glycolic acid” includes glycolide. Most preferred are poly(lactide-co-glycolide)copolymers, commonly referred to as PLGA. The polymer may have a monomer ratio of lactic acid/glycolic acid of from about 100:0 to about 15:85, preferably from about 60:40 to about 75:25 and an especially useful copolymer has a monomer ratio of lactic acid/glycolic acid of about 50:50.

As indicated in aforementioned U.S. Pat. No. 5,242,910, the polymer can be prepared in accordance with the teachings of U.S. Pat. No. 4,443,340. Alternatively, the lactic acid-based polymer can be prepared directly from lactic acid or a mixture of lactic acid and glycolic acid (with or without a further comonomer) in accordance with the techniques set forth in U.S. Pat. No. 5,310,865. The contents of all of these patents are incorporated by reference. Suitable lactic acid-based polymers are available commercially.

Examples of polymers include, but are not limited to, Poly (D,L-lactide-co-glycolide) 50:50 Resomer® RG502, code 0000366, Poly (D,L-lactide-co-glycolide) 50:50 Resomer® RG502H, PLGA-502H, code no. 260187, Poly D,L Lactide (Resomer® R 202, Resomer® R 203); Poly dioxanone (Resomer® X 210) (Boehringer Ingelheim Chemicals, Inc., Petersburg, Va.).

Additional examples include, but are not limited to, DL-lactide/glycolide 100:0 (MEDISORB® Polymer 100 DL High, MEDISORB® Polymer 100 DL Low); DL-lactide/glycolide 85/15 (MEDISORB® Polymer 8515 DL High, MEDISORB® Polymer 8515 DL Low); DL-lactide/glycolide 75/25 (MEDISORB® Polymer 7525 DL High, MEDISORB® Polymer 7525 DL Low); DL-lactide/glycolide 65/35 (MEDISORB® Polymer 6535 DL High, MEDISORB® Polymer 6535 DL Low); DL-lactide/glycolide 54/46 (MEDISORB® Polymer 5050 DL High, MEDISORB® Polymer 5050 DL Low); and DL-lactide/glycolide 54/46 (MEDISORB® Polymer 5050 DL 2A(3), MEDISORB® Polymer 5050 DL 3A(3), MEDISORB® Polymer 5050 DL 4A(3)) (Medisorb Technologies International L.P., Cincinatti, Ohio); and Poly D,L-lactide-co-glycolide 50:50; Poly D,L-lactide-co-glycolide 65:35; Poly D,L-lactide-co-glycolide 75:25; Poly D,L-lactide-co-glycolide 85:15; Poly DL-lactide; Poly L-lactide; Poly glycolide; Poly ε-caprolactone; Poly DL-lactide-co-caprolactone 25:75; and Poly DL-lactide-co-caprolactone 75:25 (Birmingham Polymers, Inc., Birmingham, Ala.).

It has been surprisingly found that injectable depot gel formulations of the invention comprising low molecular weight polymers provide a controlled, sustained release of a beneficial agent over a short duration of time equal to or less than two weeks. The release rate profile can be controlled by the appropriate choice of a low molecular weight polymer, a water immiscible solvent, the polymer/solvent ratio, emulsifying agent, thixotropic agent, pore former, solubility modifier for the beneficial agent, an osmotic agent, and the like.

The biocompatible polymer is present in the gel composition in an amount ranging from about 5 to about 90% by weight, preferably from about 10 to about 85% by weight, preferably from about 15 to about 80% by weight, preferably from about 20 to about 75% by weight, preferably from about 30 to about 70% by weight and typically from about 35 to about 65%, and often about 40 to about 60% by weight of the viscous gel, the viscous gel comprising the combined amounts of the biocompatible polymer and the solvent. The solvent will be added to polymer in amounts described below, to provide injectable depot gel compositions.

Solvents and Agents:

The injectable depot composition of the invention contains a water-immiscible solvent in addition to the bioerodible polymer and the beneficial agent. In preferred embodiments, the compositions described herein are also free of solvents having a miscibility in water that is greater than 7 wt. % at 25° C.

The solvent must be biocompatible, should form a viscous gel with the polymer, and restrict water uptake into the implant. The solvent may be a single solvent or a mixture of solvents exhibiting the foregoing properties. The term “solvent”, unless specifically indicated otherwise, means a single solvent or a mixture of solvents. Suitable solvents will substantially restrict the uptake of water by the implant and may be characterized as immiscible in water, i.e., having a solubility in water of less than 7% by weight. Preferably, the solvents are five weight percent or less soluble in water; more preferably three weight percent or less soluble in water; and even more preferably one weight percent or less soluble in water. Most preferably the solubility of the solvent in water is equal to or less than 0.5 weight percent.

Water miscibility may be determined experimentally as follows: Water (1-5 g) is placed in a tared clear container at a controlled temperature, about 20° C., and weighed, and a candidate solvent is added dropwise. The solution is swirled to observe phase separation. When the saturation point appears to be reached, as determined by observation of phase separation, the solution is allowed to stand overnight and is re-checked the following day. If the solution is still saturated, as determined by observation of phase separation, then the percent (w/w) of solvent added is determined. Otherwise more solvent is added and the process repeated. Solubility or miscibility is determined by dividing the total weight of solvent added by the final weight of the solvent/water mixture. When solvent mixtures are used, for example 20% triacetin and 80% benzyl benzoate, they are pre-mixed prior to adding to the water.

Solvents useful in this invention are generally less than 7% water soluble by weight as described above. Solvents having the above solubility parameter may be selected from aromatic alcohols, the lower alkyl and aralkyl esters of aryl acids such as benzoic acid, the phthalic acids, salicylic acid, lower alkyl esters of citric acid, such as triethyl citrate and tributyl citrate and the like, and aryl, aralkyl and lower alkyl ketones.

Many of the solvents useful in the invention are available commercially (Aldrich Chemicals, Sigma Chemicals) or may be prepared by conventional esterification of the respective arylalkanoic acids using acid halides, and optionally esterification catalysts, such as described in U.S. Pat. No. 5,556,905, which is incorporated herein by reference, and in the case of ketones, oxidation of their respective secondary alcohol precursors.

Preferred solvents include aromatic alcohols, the lower alkyl and aralkyl esters of the aryl acids described above. Representative acids are benzoic acid and the phthalic acids, such as phthalic acid, isophthalic acid, and terephathalic acid. Most preferred solvents are benzyl alcohol and derivatives of benzoic acid and include, but are not limited to, methyl benzoate, ethyl benzoate, n-propyl benzoate, isopropyl benzoate, butyl benzoate, isobutyl benzoate, sec-butyl benzoate, tert-butyl benzoate, isoamyl benzoate and benzyl benzoate, with benzyl benzoate being most especially preferred.

The composition may also include, in addition to the water-immiscible solvent(s), one or more additional miscible solvents (“component solvents”), provided that any such additional solvent is other than a lower alkanol. Component solvents compatible and miscible with the primary solvent(s) may have a higher miscibility with water and the resulting mixtures may still exhibit significant restriction of water uptake into the implant. Such mixtures will be referred to as “component solvent mixtures.” Useful component solvent mixtures may exhibit solubilities in water greater than the primary solvents themselves, typically between 0.1 weight percent and up to and including 50 weight percent, preferably up to and including 30 weight percent, and most preferably up to an including 10 weight percent, without detrimentally affecting the restriction of water uptake exhibited by the implants of the invention.

Component solvents useful in component solvent mixtures are those solvents that are miscible with the primary solvent or solvent mixture, and include, but are not limited, to triacetin, diacetin, tributyrin, triethyl citrate, tributyl citrate, acetyl triethyl citrate, acetyl tributyl citrate, triethylglycerides, triethyl phosphate, diethyl phthalate, diethyl tartrate, mineral oil, polybutene, silicone fluid, glylcerin, ethylene glycol, polyethylene glycol, octanol, ethyl lactate, propylene glycol, propylene carbonate, ethylene carbonate, butyrolactone, ethylene oxide, propylene oxide, N-methyl-2-pyrrolidone, 2-pyrrolidone, glycerol formal, methyl acetate, ethyl acetate, methyl ethyl ketone, dimethylformamide, dimethyl sulfoxide, tetrahydrofuran, caprolactam, decylmethylsulfoxide, oleic acid, and 1-dodecylazacyclo-heptan-2-one, and mixtures thereof.

The solvent or solvent mixture is capable of dissolving the polymer to form a viscous gel that can maintain particles of the beneficial agent dissolved or dispersed and isolated from the environment of use prior to release. The compositions of the present invention provide implants useful both for systemic and local administration of beneficial agent, the implants having a low burst index. Water uptake is controlled by the use of a solvent or component solvent mixture that solubilizes or plasticizes the polymer but substantially restricts uptake of water into implant. Additionally, the preferred compositions may provide viscous gels that have a glass transition temperature that is less than 37° C., such that the gel remains non-rigid for a period of time after implantation of 24 hours or more.

Compositions intended for local delivery of beneficial agent are formed in the same manner as those intended for systemic use. However, because local delivery of beneficial agent to a subject will not result in detectable plasma levels of beneficial agent, such systems have to be characterized by a percentage of beneficial agent released in a predetermined initial period, rather than a burst index as defined herein. Most typically, that period will be the first 24 hours after implantation and the percentage will be equal to the amount by weight of the beneficial agent released in the period (e.g. 24 hours) divided by the amount by weight of the beneficial agent intended to be delivered in the duration of the delivery period; multiplied by the number 100. Compositions of the present invention will have initial bursts of 40% or less, preferably 30% or less, most preferably 20% or less, for most applications.

In many instances, it may be desirable to reduce the initial burst of beneficial agent during local administration to prevent adverse effects. For example, implants of the invention containing chemotherapeutic agents are suitable for direct injection into tumors. However, many chemotherapeutic agents may exhibit toxic side effects when administered systemically. Consequently, local administration into the tumor may be the treatment method of choice. It is necessary, however, to avoid administration of a large burst of the chemotherapeutic agent if it is possible that such agent would enter the vascular or lymphatic systems where it may exhibit side affects. Accordingly, in such instances the implantable systems of the present invention having limited burst as described herein are advantageous.

In terms of efficacy ratios, for post-surgical pain management, it is usually desired to deliver a drug to achieve a sufficiently high Cₘₐₓ of the beneficial agent, e.g. an anesthetic agent, to control the pain almost immediately and then maintain a sustained level of anesthetic over a certain duration. In this instance, a higher efficacy ratio may be desirable. In other situations, however, to reduce potential side effects from a high dosage of a drug, it may be useful to maintain a tightly controlled level of active agent either in systemic circulation or distribution in the local tissues. For this type of situation, a lower efficacy ratio may be desirable. As such, because of varying patient and therapy needs, it is desirable to control the efficacy ratio of a drug delivery dosage form.

Beneficial Agents:

Although there is no limit to the anesthetics that are suitable for use as beneficial agents in the present invention, U.S. Pat. No. 6,432,986 incorporated herein by reference provides several examples, in one aspect of the present invention, the anesthetic is selected from the group consisting of: bupivacaine, levo-bupivacaine, ropivacaine, levo-ropivacaine, tetracaine, etidocaine, levo-etidocaine, dextro-etidocaine, levo-etidocaine, dextro-etidocaine, levo-mepivacaine, and combinations thereof. In other aspects, the anesthetic comprises bupivacaine.

The beneficial agent is preferably incorporated into the viscous gel formed from the polymer and the solvent in the form of particles typically having an average particle size of from about 5 to about 250 microns, preferably from about 20 to about 125 microns and often from 38 to 63 microns.

To form a suspension or dispersion of particles of the beneficial agent in the viscous gel formed from the polymer and the solvent, any conventional low shear device can be used such as a Ross double planetary mixer at ambient conditions. In this manner, efficient distribution of the beneficial agent can be achieved substantially without degrading the beneficial agent.

The beneficial agent is typically dissolved or dispersed in the composition in an amount of from about 0.1% to about 50% by weight, preferably in an amount of from about 0.5% to about 40%, more preferably in an amount of about 1% to about 30%, and often 2 to 20% by weight of the combined amounts of the polymer, solvent, and beneficial agent. Depending on the amount of beneficial agent present in the composition, one can obtain different release profiles and burst indices. More specifically, for a given polymer and solvent, by adjusting the amounts of these components and the amount of the beneficial agent, one can obtain a release profile that depends more on the degradation of the polymer than the diffusion of the beneficial agent from the composition or vice versa. In this respect, at lower beneficial agent loading, one generally obtains a release profile reflecting degradation of the polymer wherein the release rate increases with time. At higher loading, one generally obtains a release profile caused by diffusion of the beneficial agent wherein the release rate decreases with time. At intermediate loading rates, one obtains combined release profiles so that if desired, a substantially constant release rate can be attained. In order to minimize burst, loading of beneficial agent on the order of 30% or less by weight of the overall gel composition, i.e., polymer, solvent and beneficial agent, is preferred, and loading of 20% or less is more preferred.

Release rates and loading of beneficial agent will be adjusted to provide for therapeutically-effective delivery of the beneficial agent over the intended sustained delivery period. Preferably, the beneficial agent will be present in the polymer gel at concentrations that are above the saturation concentration of beneficial agent in water to provide a drug reservoir from which the beneficial agent is dispensed. While the release rate of beneficial agent depends on the particular circumstances, such as the beneficial agent to be administered, release rates on the order of from about 0.1 to about 100 micrograms/day, preferably from about 1 to about 10 micrograms per day, for periods of from about 3 days to about two weeks can be obtained. Greater amounts may be delivered if delivery is to occur over shorter periods. Generally, higher release rate is possible if a greater burst can be tolerated. In instances where the gel composition is surgically implanted, or used as a “leave behind” depot when surgery to treat the disease state or another condition is concurrently conducted, it is possible to provide higher doses that would normally be administered if the implant was injected. Further, the dose of beneficial agent may be controlled by adjusting the volume of the gel implanted or the injectable gel injected.

II. Utility and Administration:

The means of administration of the depot gel compositions is not limited to injection, although that mode of delivery may often be preferred. Where the depot gel composition will be administered as a leave-behind product, it may be formed to fit into a body cavity existing after completion of surgery or it may be applied as a flowable gel by brushing or palleting the gel onto residual tissue or bone. Such applications may permit loading of beneficial agent in the gel above concentrations typically present with injectable compositions.

Compositions of this invention without beneficial agent are useful for wound healing, bone repair and other structural support purposes.

To further understand the various aspects of the present invention, the results set forth in the previously described figures were obtained in accordance with the following examples.

### EXAMPLES

Below are several examples of specific embodiments for carrying out the present invention. The examples are offered for illustrative purposes only, and are not intended to limit the scope of the present invention in any way.

### Example 1

Depot Gel Preparation

A gel vehicle for use in an injectable depot of the composition was prepared as follows. A glass vessel was tared on a Mettler PJ3000 top loader balance. Poly (D,L-lactide-co-glycolide) (PLGA), available as 50:50 DL-PLG with an inherent viscosity of 0.15 (PLGA-BPI, Birmingham Polymers, Inc., Birmingham, Ala.) and 50:50 Resomer® RG502 (PLGA RG 502), was weighed into the glass vessel. The glass vessel containing the polymer was tared and the corresponding solvent was added. Amounts expressed as percentages for various polymer/solvent combinations are set forth in Table 1, below. The polymer/solvent mixture was stirred at 250±50 rpm (IKA electric stirrer, IKH-Werke GmbH and Co., Stanfen, Germany) for about 5-10 minutes, resulting in a sticky paste-like substance containing polymer particles. The vessel containing the polymer/solvent mixture was sealed and placed in a temperature controlled incubator equilibrated to 37° C. for 1 to 4 days, with intermittent stirring, depending on solvent and polymer type and solvent and polymer ratios. The polymer/solvent mixture was removed from the incubator when it appeared to be a clear amber homogeneous solution. Thereafter, the mixture was placed in an oven (65° C.) for 30 minutes. It was noted that the PLGA was dissolved in the mixture upon removal from the oven.

Additional depot gel vehicles are prepared with the following solvents or mixtures of solvents: benzyl benzoate (“BB”), benzyl alcohol (“BA”), ethyl benzoate (“EB”), BB/BA, BB/Ethanol, BB/EB and the following polymers: Poly (D,L-lactide) Resomer® L104, PLA-L104, code no. 33007, Poly (D,L-lactide-co-glycolide) 50:50 Resomer® RG502, code 0000366, Poly (D,L-lactide-co-glycolide) 50:50 Resomer® RG502H, PLGA-502H, code no. 260187, Poly (D,L-lactide-co-glycolide) 50:50 Resomer® RG503, PLGA-503, code no. 0080765, Poly (D,L-lactide-co-glycolide) 50:50 Resomer® RG755, PLGA-755, code no. 95037, Poly L-Lactide MW 2,000 (Resomer® L 206, Resomer® L 207, Resomer® L 209, Resomer® L 214); Poly D,L Lactide (Resomer® R 104, Resomer® R 202, Resomer® R 203, Resomer® R 206, Resomer® R 207, Resomer® R 208); Poly L-Lactide-co-D,L-lactide 90:10 (Resomer® LR 209); Poly D-L-lactide-co-glycolide 75:25 (Resomer® RG 752, Resomer®O RG 756); Poly D,L-lactide-co-glycolide 85:15 (Resomer® RG 858); Poly L-lactide-co-trimethylene carbonate 70:30 (Resomer® LT 706); Poly dioxanone (Resomer® X 210) (Boehringer Ingelheim Chemicals, Inc., Petersburg, Va.); DL-lactide/glycolide 100:0 (MEDISORB® Polymer 100 DL High, MEDISORB® Polymer 100 DL Low); DL-lactide/glycolide 85/15 (MEDISORB® Polymer 8515 DL High, MEDISORB® Polymer 8515 DL Low); DL-lactide/glycolide 75/25 (MEDISORB® Polymer 7525 DL High, MEDISORB® Polymer 7525 DL Low); DL-lactide/glycolide 65/35 (MEDISORB® Polymer 6535 DL High, MEDISORB® Polymer 6535 DL Low); DL-lactide/glycolide 54/46 (MEDISORB® Polymer 5050 DL High, MEDISORB® Polymer 5050 DL Low); and DL-lactide/glycolide 54/46 (MEDISORB® Polymer 5050 DL 2A(3), MEDISORB® Polymer 5050 DL 3A(3), MEDISORB® Polymer 5050 DL 4A(3)) (Medisorb Technologies International L.P., Cincinnati, Ohio); and Poly D,L-lactide-co-glycolide 50:50; Poly D,L-lactide-co-glycolide 65:35; Poly D,L-lactide-co-glycolide 75:25; Poly D,L-lactide-co-glycolide 85:15; Poly DL-lactide; Poly L-lactide; Poly glycolide; Poly ε-caprolactone; Poly DL-lactide-co-caprolactone 25:75; and Poly DL-lactide-co-caprolactone 75:25 (Birmingham Polymers, Inc., Birmingham, Ala.).

### Example 2

Bupivacaine Base Preparation

Bupivacaine hydrochloride (Sigma-Aldrich Corporation, St. Louis, Mo.) was dissolved in de-ionized (DI) water at a concentration of 40 mg/ml (saturation). A calculated amount of sodium hydroxide (1 N solution) was added to the solution and the pH of the final mixtures was adjusted to 10 to precipitate the BP base. The precipitated product was filtered, and further washed with DI water for at least three times. The precipitated product was dried at approximately 40° C. in vacuum for 24 hours.

### Example 3

Bupivacaine Particle Preparation

Bupivacaine drug particles using bupivacaine hydrochloride (Sigma-Aldrich Corporation, St. Louis, Mo.) or bupivacaine base prepared according example 4 and hydrochloride salt, were prepared as follows. Bupivicaine was grounded and then sieved to a fixed range using 3″ stainless steel sieves. Typical ranges included 25 μm to 38 μm, 38 μm to 63 μm, and 63 μm to 125 μm.

### Example 4

Bupivacaine-Stearic Acid Particle Preparation

Bupivacaine particles were prepared as follows: Bupivacaine hydrochloride (100 g, Sigma-Aldrich Corporation, St. Louis, Mo.) was grounded and sieved through 63-125 micron sieves. The bupivacaine particles and stearic acid (100 g, 95% pure, Sigma-Aldrich Corporation, St. Louis, Mo.) were blended and ground. The ground material was compressed in a 13 mm round die, with a force of 5,000 pounds for 5 minutes. Compressed tablets were ground and sieved through a 120 mesh screen followed by a 230 mesh screen to obtain particles having a size between 63-125 microns.

### Example 5

Drug Loading

Particles comprising beneficial agent with or without stearic acid prepared as above were added to a gel vehicle in an amount of 10-30% by weight and blended manually until the dry powder was wetted completely. Then, the milky light yellow particle/gel mixture was thoroughly blended by conventional mixing using a Caframo mechanical stirrer with an attached square-tip metal spatula. Resulting formulations are illustrated in Tables 1-3 below.

TABLE 1

| | PLGA RG502^{a} | LMW PLGA^{b} | Benzyl Benzoate |
|---|---|---|---|
| Formulation | (wt %) | (wt %) | (wt %) |
| 1^{c} | 45 | 0 | 45 |
| 2^{c} | 0 | 45 | 45 |
| 3^{d} | 45 | 0 | 45 |
| 4^{d} | 0 | 45 | 45 |

^{a}= PLGA RG 502, MW = 16,000.
^{b}= Low Molecular Weight (LMW, MW = 8,000) PLGA with an ester end group.
^{c}= 10% bupivacaine hydrochloride loading.
^{d}= 10% bupivacaine base loading.

TABLE 2

| | | | Benzyl | Benzyl |
|---|---|---|---|---|
| | LMW PLGA^{f} | LMW PLGAc^{g} | Benzoate | Alcohol |
| Formulation | (wt %) | (wt %) | (wt %) | (wt %) |
| 5^{h} | 58.5 | 0 | 31.5 | 0 |
| 6^{h} | 58.5 | 0 | 0 | 31.5 |
| 7^{h} | 67.5 | 0 | 0 | 22.5 |
| 8^{h} | 0 | 67.5 | 0 | 22.5 |
| 9^{f} | 0 | 60 | 0 | 20 |

^{f}= Low Molecular Weight (LMW, MW = 8,000) PLGA with an ester end group.
^{g}= Low Molecular Weight (LMW, MW = 10,000) PLGA with a carboxyl end group.
^{h}= 10% bupivacaine hydrochloride loading.
ⁱ= 10% bupivacaine hydrochloride and 10% SA loading.

TABLE 3

| | LMW PLGAc^{j} | Benzyl Benzoate | Benzyl Alcohol |
|---|---|---|---|
| Formulation | (wt %) | (wt %) | (wt %) |
| 10^{k} | 52.5 | 0 | 17.5 |
| 11^{l} | 52.5 | 0 | 17.5 |
| 12^{k} | 45.5 | 0 | 24.5 |
| 13^{k} | 45.5 | 12.3 | 12.3 |

^{j}= Low Molecular Weight (LMW, MW = 10,000) PLGA with a carboxyl end group.
^{k}= 30% bupivacaine hydrochloride loading, particle size between 63-125 μm.
^{l}= 30% bupivacaine hydrochloride loading, particle size between 38-63 μm.

A representative number of implantable depots gel compositions were prepared in accordance with the foregoing procedures and tested for in vitro release of beneficial agent as a function of time and also in in vivo studies in rats to determine release of the beneficial agent as determined by blood plasma concentrations of beneficial agent as a function of time.

A representative number of implantable depots gel compositions are also prepared in accordance with the foregoing procedures and are tested in in vivo studies in rats to determine local release of the beneficial agent as determined by local tissue sampling as a function of time.

### Example 6A

Bupivacaine In Vivo Studies

In vivo studies in rats (4 or 5 per group) were performed following an open protocol to determine plasma levels of bupivacaine upon systemic administration of bupivacaine via the implant systems of this invention. Depot gel bupivacaine formulations were loaded into customized 0.5 cc disposable syringes. Disposable 18 gauge needles were attached to the syringes and were heated to 37° C. using a circulator bath. Depot gel bupivacaine formulations were injected into rats and blood was drawn at specified time intervals (1 hour, 4 hours and on days 1, 2, 5, 7, 9,14, 21 and 28) and analyzed for bupivacaine using LC/MS.

### Example 6B

Bupivacaine Local Administration Studies

In vivo studies in rats (4 or 5 per group) are performed following an open protocol to determine plasma levels of bupivacaine upon local administration of bupivacaine via the implant systems of this invention. Depot gel bupivacaine formulations are loaded into customized 0.5 cc disposable syringes. Disposable 18 gauge needles are attached to the syringes and are heated to 37° C. using a circulator bath. Depot gel bupivacaine formulations are injected into rats and local tissue is sampled at specified time intervals (1 hour, 4 hours and on days 1, 2, 5, 7, 9,14, 21 and 28) and is homogenized. The bupivacaine in the local tissue is extracted and analyzed using LC/MS.

### Example 7

Bupivacaine Release for Short Durations

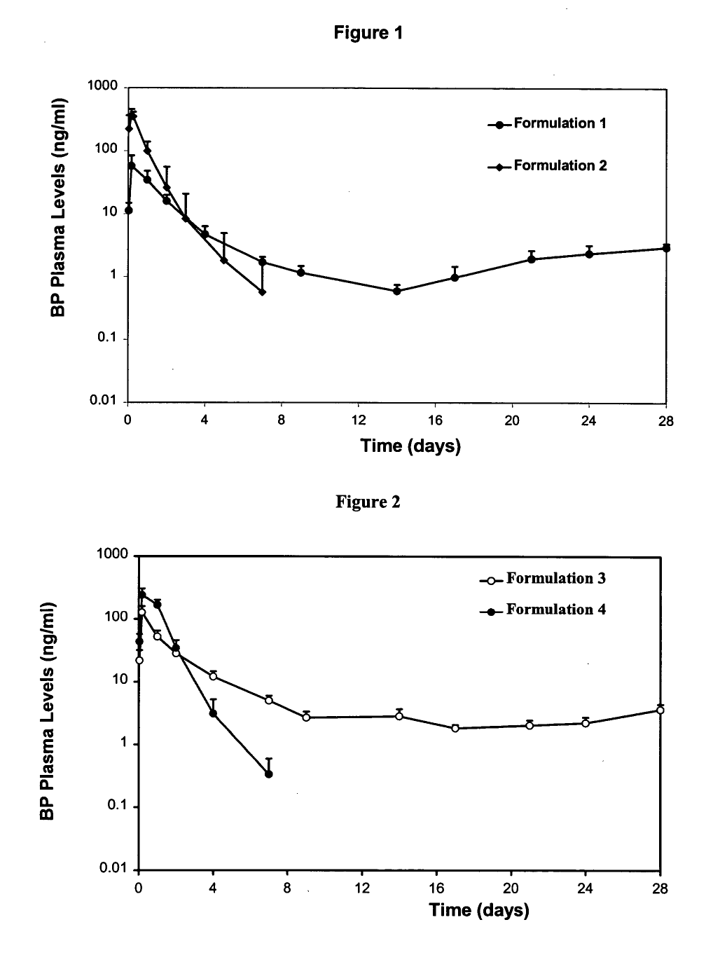

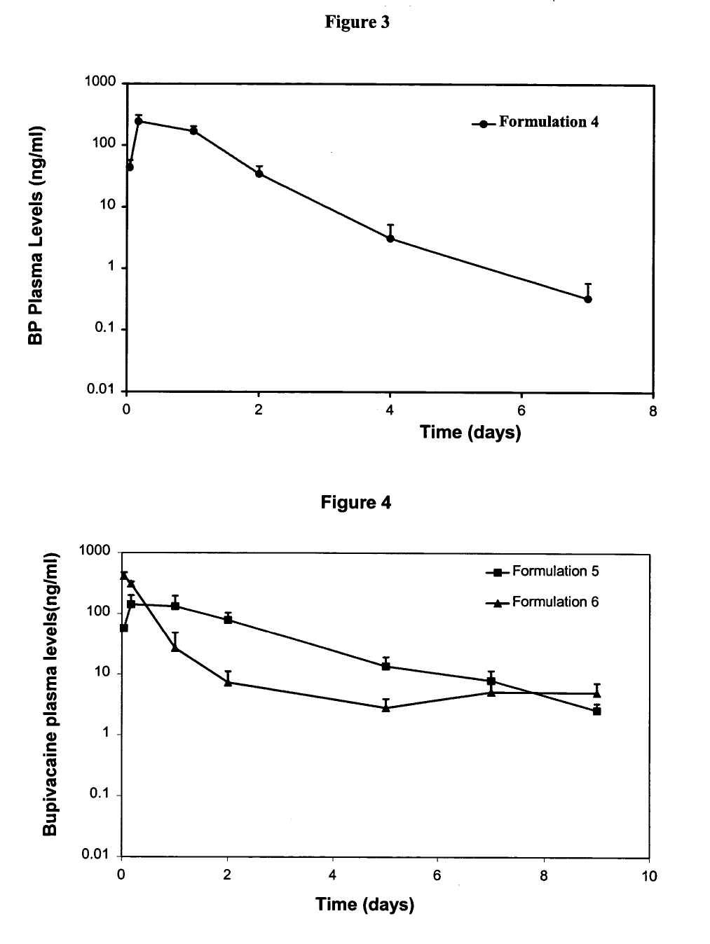

FIGS. 1, 2 and 3 illustrate representative in vivo release profiles of bupivacaine hydrochloride and bupivacaine base obtained in rats from various depot formulations, including those of the present invention. The in vivo release profile of the depot formulations with low molecular weight PLGA (formulations 2 and 4 in FIGS. 1, 2 and 3) exhibited short release duration for approximately 7 days, comparable to the control formulations (with higher molecular weight PLGA). Thus, the injectable depot gel formulations of the invention comprising low molecular weight polymers provide a controlled, sustained release of a beneficial agent over a short duration of time equal to or less than two weeks.

As illustrated in Tables 2 & 3 and FIGS. 1-12, various depot formulations can be made from the low molecular weight PLGA with either an ester end group or a carboxyl end group using different solvents such as benzyl benzoate (BB), benzyl alcohol (BA), ethyl benzoate (EB), mixtures of BB/Ethanol, BB/BA, BB/EB etc., with varying polymer/solvent ratios, drug loadings and drug forms. The drug particles can be made either with or without hydrophobic excipients such as stearic acid (SA).

### Example 8

Effect of Solvent on the Bupivacaine Release

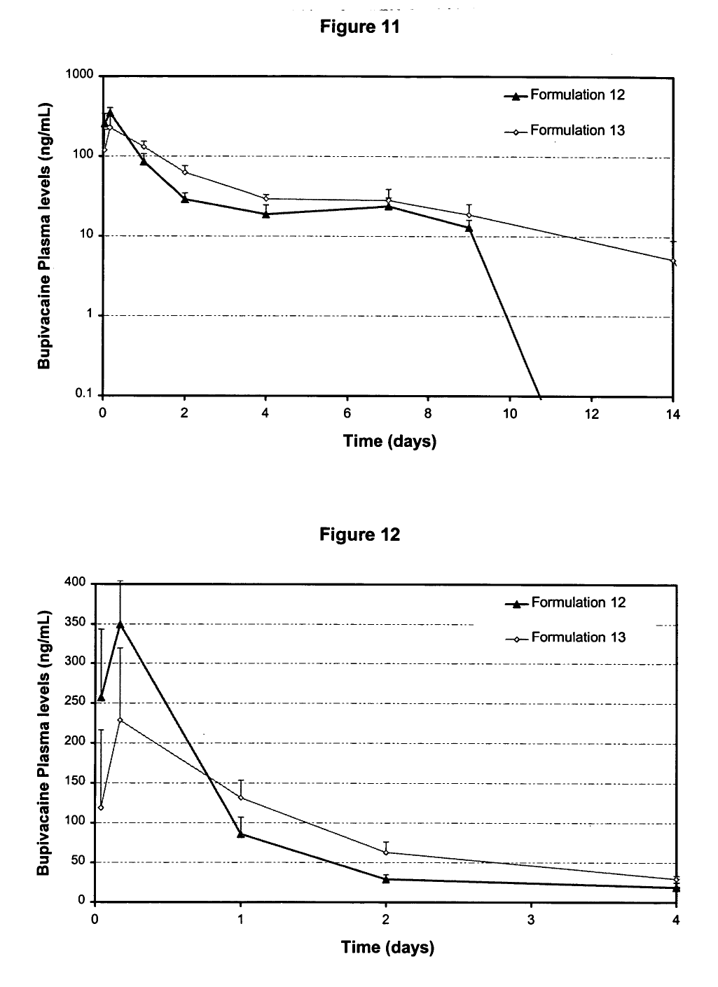

FIG. 4 illustrates representative in vivo release profiles of bupivacaine obtained in rats from depot formulations made of low molecular weight PLGA in either BB or BA (formulations 5 and 6). FIGS. 11 & 12 illustrate representative in vivo release profiles of bupivacaine obtained in rats from depot formulations made of low molecular weight PLGA in either BA or mixture of BA with BB (BA/BB, 50/50) (formulations 12 and 13). The release rate profiles of bupivacaine from such short duration depots can be altered and controlled by the solvent used in the formulations. As summarized in Table 4, the Cₘₐₓ, C_{average} and the efficacy ratio (Cₘₐₓ/C_{average}) can be affected by the solvent used in the depot formulations.

TABLE 4

| Formulation | Cₘₐₓ^{a} | C_{average}^{b} | Efficacy Ratio |
|---|---|---|---|
| 5^{c} | 147 ± 51 | 26 ± 34 | 5.7 |
| 6^{c} | 417 ± 53 | 5 ± 3 | 83.4 |
| 12^{d} | 350 ± 55 | 21 ± 8 | 16.6 |
| 13^{d} | 229 ± 90 | 29 ± 21 | 7.9 |

^{a}= Cₘₐₓ = maximum plasma concentration of bupivacaine;
^{b}= C_{average} = average plasma concentration of bupivacaine from day 2 to day 9;
^{c}= 10% bupivacaine hydrochloride loading;
^{d}= 30% bupivacaine hydrochloride loading.

### Example 9

Effect of Polymer/Solvent Ratios on the Bupivacaine Release

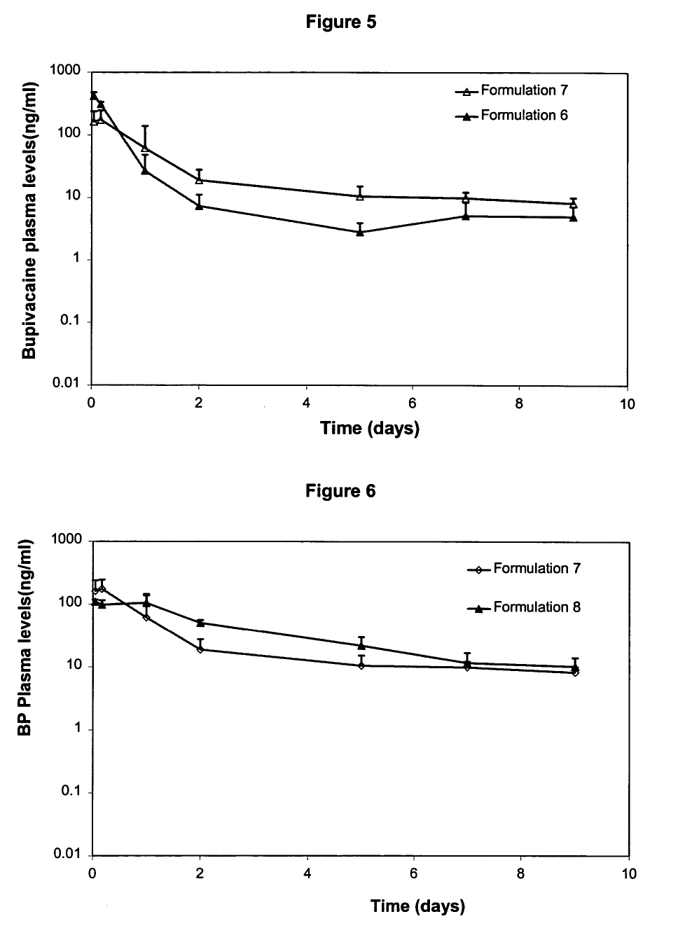

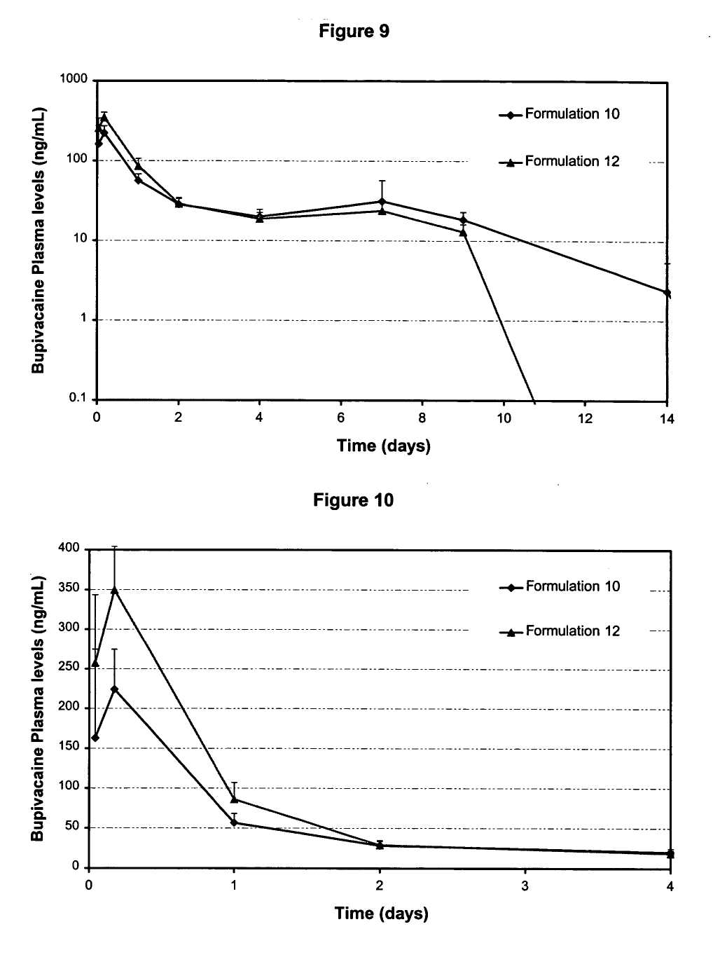

FIG. 5 illustrates representative in vivo release profiles of bupivacaine obtained in rats from depot formulations made of low molecular weight PLGA having an ester end group in BA with various polymer/solvent ratios (formulations 6 and 7). FIGS. 9 and 10 illustrate representative in vivo release profiles of bupivacaine obtained in rats from depot formulations made of low molecular weight PLGA having carboxyl group in BA with various polymer/solvent ratios (formulations 10 and 12). The release rate profiles of bupivacaine from such short duration depots can be altered and controlled by the polymer/solvent ratios in the formulations. As summarized in Table 5, the Cₘₐₓ, C_{average} and the efficacy ratio (Cₘₐₓ/C_{averge}) can be affected by the polymer/solvent ratios in the depot formulations.

TABLE 5

| Formulation | Cₘₐₓ^{a} | C_{average}^{b} | Efficacy Ratio |
|---|---|---|---|
| 6^{c} | 417 ± 53 | 5 ± 3 | 83.4 |
| 7^{c} | 177 ± 62 | 12 ± 6 | 14.8 |
| 10^{d} | 235 ± 72 | 25 ± 13 | 9.6 |
| 12^{d} | 350 ± 55 | 21 ± 8 | 16.6 |

^{a}= Cₘₐₓ = maximum plasma concentration of bupivacaine;
^{b}= C_{average} = average plasma concentration of bupivacaine from day 2 to day 9;
^{c}= 10% bupivacaine hydrochloride loading, LMW PLGA with an ester end group, MW = 8,000;
^{d}= 30% bupivacaine hydrochloride loading, LMW PLGA with a carboxyl end group, MW = 10,000.

### Example 10

Effect of Drug Excipient on the Bupivacaine Release

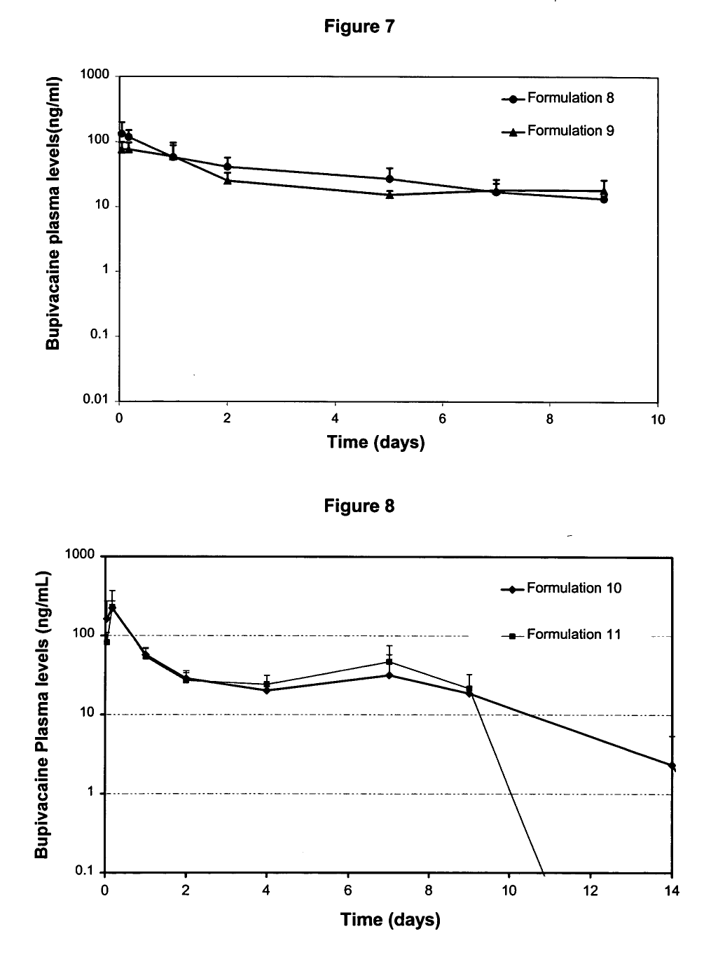

FIG. 7 illustrates representative in vivo release profiles of bupivacaine obtained in rats from depot formulations made of low molecular weight PLGA in BA with the drug particles formulated either with or without SA (formulation 8 and 9). The release rate profiles of bupivacaine from such short duration depots can be altered and controlled by drug excipient used in the formulations. As summarized in Table 6, the Cₘₐₓ, C_{average} and the efficacy ratio (Cₘₐₓ/C_{average}) can be affected by drug excipient such as stearic acid used in the depot formulations.

TABLE 6

| Formulation | Cₘₐₓ^{a} | C_{average}^{b} | Efficacy Ratio |
|---|---|---|---|
| 8^{c} | 128 ± 22 | 24 ± 18 | 5.3 |
| 9^{d} | 79 ± 22 | 17 ± 6 | 4.6 |

^{a}= Cₘₐₓ = maximum plasma concentration of bupivacaine;
^{b}= C_{average} = average plasma concentration of bupivacaine from day 2 to day 9;
^{c}= 10% bupivacaine hydrochloride loading, LMW PLGA with a carboxyl end group, MW = 10,000;
^{d}= 20% loading with 10% bupivacaine hydrochloride compacted with 10% stearic acid, LMW PLGA with a carboxyl end group, MW = 10,000.

### Example 11

Differential Scanning Calorimeter (DSC) Measurements on PLGA Polymers

The glass transition temperature of various low molecular PLGA polymers used in the present invention was determined using a differential scanning calorimeter (DSC) (Perkin Elmer Pyris 1, Shelton, Conn.). The DSC sample pan was tarred on a Mettler PJ3000 to s loader balance. At least 20 mg of polymer sample was placed in the pan. The weight of the sample was recorded. The DSC pan cover was positioned on to the pan and a presser was used to seal the pan. The temperature was scanned in 10° C. increments from −50° C. to 90° C.

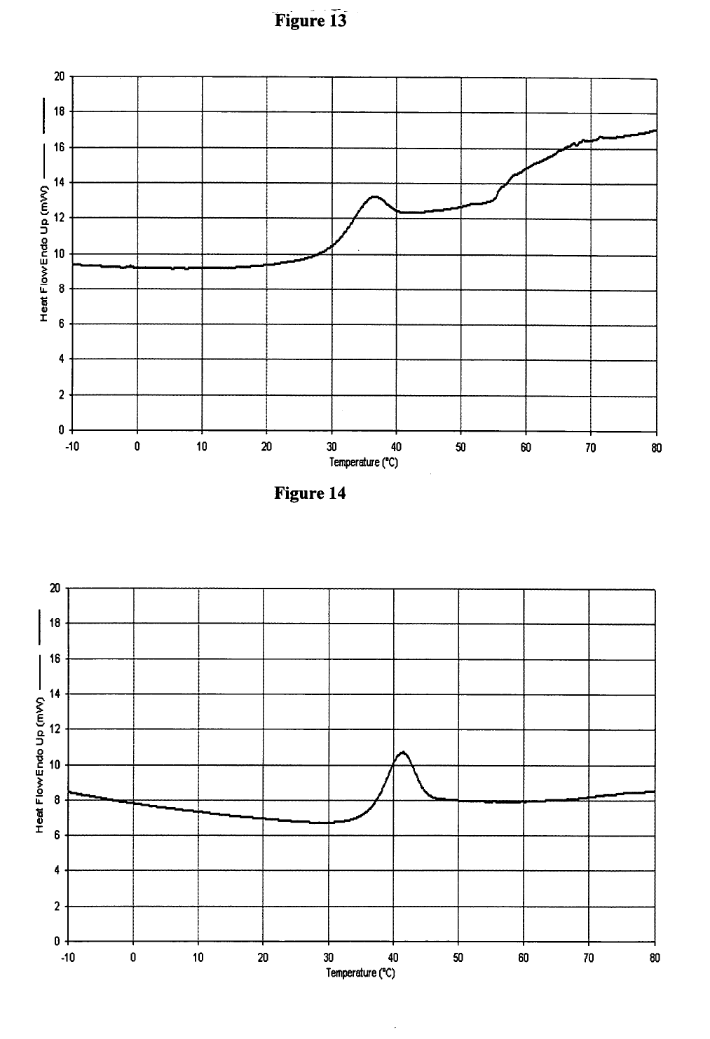

FIGS. 13 and 14 illustrate the differences in the DSC diagrams of low molecular weight PLGA used in the formulations presented in this invention end-capped with either an ester group or the carboxyl terminated. FIG. 13 shows a DSC diagram of low molecular weight PLGA (L/G ratio 50/50, MW=8,000) with an ester end group. FIG. 14 shows a DSC diagram of low molecular weight PLGA (L/G ratio 50/50, MW=10,000) with carboxyl end group. These data demonstrate that the low molecular weight PLGA polymers used in this invention have a glass transition temperatures (“Tg”) above 30° C.

### Example 12

In Vitro Degradation of PLGA Polymers

The degradation profiles of low molecular weight PLGA polymers used in the present invention were performed in vitro at 37° C. in PBS buffer to determine the mass loss rate of the PLGA polymer as a function of time. Each of the copolymers comprised one sample set. Approximately 25 discs (100±5 mg each) were pressed using a 13 mm stainless steel die. The sample was pressed with 10 tons of force for approximately 10 minutes using the Carver Press. The discs were kept in a glass vial in a vacuum oven at ambient temperature and 25 mm Hg until ready for use in the degradation bath. This procedure was repeated for each polymer tested. Phosphate buffered saline (PBS) solution (50 mM, pH 7.4) with sodium azide (0.1N ) was prepared. One sample disc was weighed into the tarred vial and recorded as initial weight (Mᵢₙᵢₜᵢₐₗ). PBS (10 mL) was pipetted into each vial. The vial was capped securely and placed in a 37° C. shaking water bath. The buffer was changed twice a week, prior to which the pH of the solution was recorded. At pre-designated time points, the samples were removed from the buffer bath, rinsed with de-ionized Milli-Q water, dried superficially, and weighed. The sample weight was recorded as wet weight (M_{wet}). The sample was placed in a 10 mL lyophilization vial and placed in a freezer (−20° C.) prior to lyophilization. After lyophilization, the samples were weighed again and recorded as dry weight (M_{lyophilized}). The percent mass loss was defined as {(M_{lyophilized}−Mᵢₙᵢₜᵢₐₗ)/Mᵢₙᵢₜᵢₐₗ}×100%.

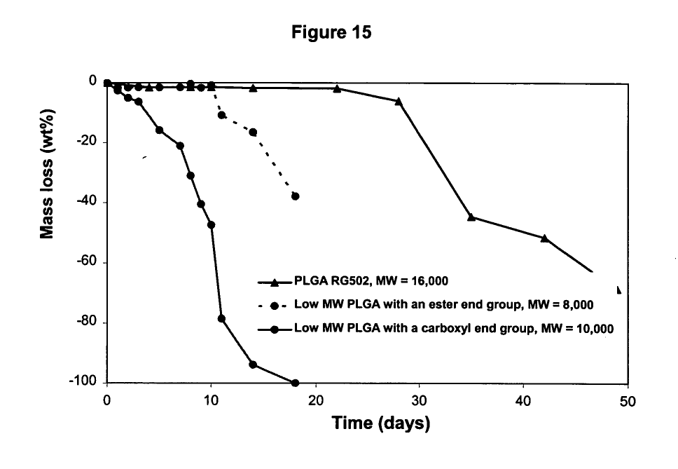

FIG. 15 illustrates the mass loss profiles of the three PLGAs used in the formulations described above. From this it can be seen that each of the three polymers used has significantly different degradation rates. The low molecular weight PLGA with either an ester end group or carboxyl end group have a significantly faster degradation rate than the one with higher molecular weight. This represents more favorable towards short duration depot which prefers the polymer degrades as soon as the active agents are released from the depot. In accordance with various aspects of the present invention, one or more significant advantages can be obtained. More specifically, using simple processing steps, one can obtain a depot gel composition that can be injected into place in an animal without surgery using a low dispensing force through standard needles. Once in place, the composition will quickly return to its original viscosity and may exhibit rapid hardening so as to substantially avoid a burst effect and provide the desired beneficial agent release profile. Furthermore, once the beneficial agent has been fully administered, there is no need to remove the composition since it is fully biodegradable. As a still further advantage, the present invention avoids the use of microparticle or microcapsulation techniques which can degrade certain beneficial agents, like peptide and nucleic acid-based drugs and which microparticles and microcapsules maybe difficult to remove from the environment of use. Since the viscous gel is formed without the need for water, temperature extremes, or other solvents, suspended particles of beneficial agent remain dry and in their original configuration, which contributes to the stability of thereof. Further, since a mass is formed, the injectable depot gel composition may be retrieved from the environment of use if desired.

### Example 13

Effect of Weight Average Molecular Weight on Bupivacaine Release

FIG. 6 illustrates representative in vivo release profiles of bupivacaine obtained in rats from depot formulations made of low molecular weight (8,000) PLGA with an ester end group in BA (formulation 7) and low molecular weight (10,000) PLGA with a carboxyl end group in BA (formulation 8). The release rate profiles of bupivacaine from such short duration depots can be altered and controlled by the molecular weight of the polymer and/or the end group in the PLGA used in the formulations.

### Example 14

Effect of Beneficial Agent Average Particle Size on Bupivacaine Release

FIG. 8 illustrates representative in vivo release profiles of bupivacaine obtained in rats from depot formulations made of low molecular weight (10,000) PLGA with a carboxyl end group in BA with average particle size of bupivacaine hydrochloride being 63-125 μm (formulation 10) and 38-63 μm (formulation 11). The release rate profiles of bupivacaine from such short duration depots can be altered and controlled by the average size of the active agent.

What is claimed:

1. A sustained release dosage form of an anesthetic comprising:
a short duration gel vehicle comprising a low molecular weight bioerodible, biocompatible polymer and a water-immiscible solvent in an amount effective to plasticize the polymer and form a gel therewith;
an anesthetic dissolved or dispersed in the gel vehicle.

2. The sustained release dosage form of claim 1 further comprising a controllable efficacy ratio to achieve a release profile.

3. The sustained release dosage form of claim 2 wherein the efficacy ratio is between about 1 and 200.

4. The sustained release dosage form of claim 3 wherein the efficacy ratio is between about 5 and 100.

5. The sustained release dosage form of claim 1 wherein the sustained release occurs in a period of less than or equal to about fourteen days.

6. The sustained release dosage form of claim 5 wherein the sustained release occurs in a period of less than or equal to about seven days.

7. The sustained release dosage form of claim 6 wherein the sustained release lasts for a period of between about 24 hours and about seven days.

8. The sustained release dosage form of claim 1 wherein the anesthetic is selected from the group consisting of: bupivacaine, levo-bupivacaine, ropivacaine, levo-ropivacaine, tetracaine, etidocaine, levo-etidocaine, dextro-etidocaine, levo-etidocaine, dextro-etidocaine, levo-mepivacaine, and combinations thereof.

9. The sustained release dosage form of claim 1 wherein the anesthetic comprises bupivacaine.

10. The sustained release dosage form of claim 1 wherein the solvent has a miscibility in water of less than or equal to about 7 weight % at 25° C.

11. The sustained release dosage form of claim 1 wherein the dosage form is free of solvents having a miscibility in water that is greater than 7 weight % at 25° C.

12. The sustained release dosage form of claim 1 wherein the solvent is selected from the group consisting of: an aromatic alcohol, lower alkyl esters of aryl acids, lower aralkyl esters of aryl acids, aryl ketones, aralkyl ketones, lower alkyl ketones, lower alkyl esters of citric acid, and combinations thereof.

13. The sustained release dosage form of claim 1 wherein the solvent comprises benzyl alcohol.

14. The sustained release dosage form of claim 1 wherein the solvent comprises benzyl benzoate.

15. The sustained release dosage form of claim 1 wherein the solvent comprises ethyl benzoate.

16. The sustained release dosage form of claim 1 wherein the solvent comprises triacetin.

17. The sustained release dosage form of claim 1 wherein the solvent comprises a component solvent selected from the group consisting of: triacetin, diacetin, tributyrin, triethyl citrate, tributyl citrate, acetyl triethyl citrate, acetyl tributyl citrate, triethylglycerides, triethyl phosphate, diethyl phthalate, diethyl tartrate, mineral oil, polybutene, silicone fluid, glylcerin, ethylene glycol, polyethylene glycol, octanol, ethyl lactate, propylene glycol, propylene carbonate, ethylene carbonate, butyrolactone, ethylene oxide, propylene oxide, N-methyl-2-pyrrolidone, 2-pyrrolidone, glycerol formal, methyl acetate, ethyl acetate, methyl ethyl ketone, dimethylformamide, dimethyl sulfoxide, tetrahydrofuran, caprolactam, decylmethylsulfoxide, oleic acid, and 1-dodecylazacyclo-heptan-2-one, and combinations thereof.

18. The sustained release dosage form of claim 1 wherein the polymer comprises a lactic acid-based polymer.

19. The sustained release dosage form of claim 18 wherein the polymer comprises a copolymer of lactic acid and glycolic acid (PLGA).

20. The sustained release dosage form of claim 19 wherein the copolymer has a monomer ratio of lactic acid to glycolic acid of approximately 50:50.

21. The sustained release dosage form of claim 1 wherein the polymer comprises a caprolactone-based polymer.

22. The sustained release dosage form of claim 1 wherein the polymer is selected from the group consisting of: polylactides, polyglycolides, poly(caprolactone), polyanhydrides, polyamines, polyesteramides, polyorthoesters, polydioxanones, polyacetals, polyketals, polycarbonates, polyphosphoesters, polyesters, polybutylene terephthalate, polyorthocarbonates, polyphosphazenes, succinates, poly(malic acid), poly(amino acids), polyvinylpyrrolidone, polyethylene glycol, polyhydroxycellulose, polysaccharides, chitin, chitosan, hyaluronic acid, and copolymers, terpolymers and mixtures thereof.

23. The sustained release dosage form of claim 19 wherein the polymer comprises an ester end group.

24. The sustained release dosage form of claim 19 wherein the polymer comprises a carboxylic acid end group.

25. The sustained release dosage form of claim 1 wherein the polymer has a weight average molecular weight of between about 3,000 and about 10,000.

26. The sustained release dosage form of claim 25 wherein the polymer has a weight average molecular weight of between about 3,000 and about 8,000.

27. The sustained release dosage form of claim 26 wherein the polymer has a weight average molecular weight of between about 4,000 and about 6,000.

28. The sustained release dosage form of claim 27 wherein the polymer has a weight average molecular weight of about 5,000.

29. The sustained release dosage form of claim 1 wherein the dosage form comprises from about 0.1% to about 50% anesthetic by weight.

30. The sustained release dosage form of claim 29 wherein the dosage form comprises from about 0.5% to about 40% anesthetic by weight.

31. The sustained release dosage form of claim 30 wherein the dosage form comprises from about 1% to about 30% anesthetic by weight.

32. The sustained release dosage form of claim 1 wherein the ratio between the polymer and the solvent is between about 5:95 and about 90:10.

33. The sustained release dosage form of claim 32 wherein the ratio between the polymer and the solvent is between about 20:80 and about 80:20.

34. The sustained release dosage form of claim 33 wherein the ratio between the polymer and the solvent is between about 30:70 and about 75:25.

35. The sustained release dosage form of claim 1 further comprising at least one of the following: an excipient, an emulsifying agent, a pore former, a solubility modulator for the anesthetic, and an osmotic agent.

36. The sustained release dosage form of claim 1 wherein the anesthetic comprises particles having an average particle size of less than about 250 μm.

37. The sustained release dosage form of claim 36 wherein the anesthetic comprises particles having an average particle size of between about 5 μm and 250 μm.

38. The sustained release dosage form of claim 37 wherein the average particle size is between about 20 μm and about 125 μm.

39. The sustained release dosage form of claim 38 wherein the average particle size is between about 38 μm and about 63 μm.

40. A sustained release dosage form of an anesthetic comprising:
a short duration gel vehicle comprising a low molecular weight lactic acid-based polymer and a water-immiscible solvent, in an amount effective to plasticize the polymer and form a gel therewith;
an anesthetic comprising bupivacaine, wherein the anesthetic is dissolved or dispersed in the gel vehicle; and
a controllable efficacy ratio to achieve a release profile;
wherein the weight average molecular weight of the lactic acid-based polymer is between about 3,000 and about 10,000.

41. The sustained release dosage form of claim 40 wherein the sustained release occurs in a period of less than or equal to about fourteen days.

42. The sustained release dosage form of claim 41 wherein the sustained release occurs in a period of less than or equal to about seven days.

43. The sustained release dosage form of claim 42 wherein the sustained release lasts for a period of between about 24 hours and about seven days.

44. The sustained release dosage form of claim 40 wherein the efficacy ratio is between about 1 and about 200.

45. The sustained release dosage form of claim 44 wherein the efficacy ratio is between about 5 and about 100.

46. The sustained release dosage form of claim 40 wherein the polymer comprises a copolymer of lactic acid and glycolic acid (PLGA).

47. The sustained release dosage form of claim 46 wherein the copolymer has a monomer ratio of lactic acid to glycolic acid of approximately 50:50.

48. The sustained release dosage form of claim 46 wherein the copolymer comprises poly(D,L-lactide-co-glycolide).

49. The sustained release dosage form of claim 46 wherein the copolymer comprises poly(L-lactide-co-glycolide).

50. The sustained release dosage form of claim 40 wherein the solvent has a miscibility in water of less than or equal to about 7 weight % at 25° C.

51. The sustained release dosage form of claim 40 wherein the dosage form is free of solvents having a miscibility in water that is greater than 7 weight % at 25° C.

52. The sustained release dosage form of claim 40 wherein the solvent is selected from the group consisting of: an aromatic alcohol, lower alkyl esters of aryl acids, lower aralkyl esters of aryl acids; aryl ketones, aralkyl ketones, lower alkyl ketones, lower alkyl esters of citric acid, and combinations thereof.

53. The sustained release dosage form of claim 40 wherein the solvent comprises benzyl alcohol.

54. The sustained release dosage form of claim 40 wherein the solvent comprises benzyl benzoate.

55. The sustained release dosage form of claim 40 wherein the solvent comprises ethyl benzoate.

56. The sustained release dosage form of claim 40 wherein the solvent comprises triacetin.

57. The sustained release dosage form of claim 40 wherein the polymer has a weight average molecular weight of between about 3,000 and 8,000.

58. The sustained release dosage form of claim 57 wherein the polymer has a weight average molecular weight of between about 4,000 and 6,000.

59. The sustained release dosage form of claim 58 wherein the polymer has a weight average molecular weight of about 5,000.

60. The sustained release dosage form of claim 40 wherein the dosage form comprises from about 0.1% to about 50% anesthetic by weight.

61. The sustained release dosage form of claim 60 wherein the dosage form comprises from about 0.5% to about 40% anesthetic by weight.

62. The sustained release dosage form of claim 61 wherein the dosage form comprises from about 1% to about 30% anesthetic by weight.

63. The sustained release dosage form of claim 62 wherein the ratio between the polymer and the solvent is between about 5:95 and about 90:10.

64. The sustained release dosage form of claim 63 wherein the ratio between the polymer and the solvent is between about 20:80 and about 80:20.

65. The sustained release dosage form of claim 64 wherein the ratio between the polymer and the solvent is between about 30:70 and about 75:25.

66. The sustained release dosage form of claim 40 wherein the anesthetic comprises particles having an average particle size of less than about 250 μm.

67. The sustained release dosage form of claim 66 wherein the anesthetic comprises particles having an average particle size of between about 5 μm and about 250 μm.

68. The sustained release dosage form of claim 67 wherein the average particle size is between about 20 μm and about 125 μm.

69. The sustained release dosage form of claim 68 wherein the average particle size is between about 38 μm and about 63 μm.

70. The sustained release dosage form of claim 46 wherein the PLGA comprises an ester end group.

71. The sustained release dosage form of claim 46 wherein the PLGA comprises a carboxyl end group.

72. The sustained release dosage form of claim 40 further comprising at least one of the following: an excipient, an emulsifying agent, a pore former, a solubility modulator for the anesthetic, and an osmotic agent.

73. A method of treating local pain of a subject using a sustained release dosage form, the method comprising:
administering a short duration sustained release dosage form comprising a gel vehicle, which comprises a low molecular weight bioerodible, biocompatible polymer, and a water-immiscible solvent in an amount effective to plasticize the polymer and form a gel therewith; and an anesthetic dissolved or dispersed in the gel vehicle.

74. The method of claim 73 wherein the sustained release dosage form further comprises a controllable efficacy ratio to achieve a release profile.

75. The method of claim 74 wherein the efficacy ratio is between about 1 and 200.

76. The method of claim 75 wherein the efficacy ratio is between about 5 and 100.

77. The method of claim 73 wherein the sustained release occurs in a period of less than or equal to about fourteen days.

78. The method of claim 77 wherein the sustained release occurs in a period of less than or equal to about seven days.

79. The method of claim 78 wherein the sustained release lasts for a period of between about 24 hours and about seven days.

80. The method of claim 73 further comprising administering the dosage form once.

81. The method of claim 73 further comprising applying the dosage form topically to the local pain.

82. The method of claim 73 further comprising injecting the dosage form at a location near the local pain.

83. The method of claim 73 further comprising delivering the anesthetic systemically.

84. The method of claim 73 further comprising delivering the anesthetic to multiple sites.

85. The method of claim 84 further comprising delivering injecting the dosage form at multiple locations surrounding the local pain.

86. The method of claim 73 further comprising repeating the administration of the dosage form.

87. The method of claim 73 wherein the anesthetic is selected from the group consisting of: bupivacaine, levo-bupivacaine, ropivacaine, levo-ropivacaine, tetracaine, etidocaine, levo-etidocaine, dextro-etidocaine, levo-etidocaine, dextro-etidocaine, levo-mepivacaine, and combinations thereof.

88. The method of claim 73 wherein the anesthetic comprises bupivacaine.

89. The method of claim 73 wherein the has a miscibility in water of less than or equal to about 7 weight % at 25° C.

90. The method of claim 73 wherein the polymer has a molecular weight of between about 3,000 and 10,000.

91. The method of claim 90 wherein the polymer has a weight average molecular weight of between about 3,000 and 8,000.

92. The method of claim 91 wherein the polymer has a weight average molecular weight of between about 4,000 and 6,000.

93. The method of claim 92 wherein the polymer has a weight average molecular weight of about 5,000.

94. The method of claim 73 wherein the dosage form comprises from about 0.1 to about 50% anesthetic by weight.

95. The method of claim 73 wherein the polymer is selected from the group consisting of: polylactides, polyglycolides, poly(caprolactone), polyanhydrides, polyamines, polyesteramides, polyorthoesters, polydioxanones, polyacetals, polyketals, polycarbonates, polyphosphoesters, polyesters, polybutylene terephthalate, polyorthocarbonates, polyphosphazenes, succinates, poly(malic acid), poly(amino acids), polyvinylpyrrolidone, polyethylene glycol, polyhydroxycellulose, polysaccharides, chitin, chitosan, hyaluronic acid, and copolymers, terpolymers and mixtures thereof.

96. The method of claim 73 wherein the sustained release dosage form comprises a ratio of about 5:95 and about 90:10 between the polymer and the solvent.

97. The method of claim 73 wherein the anesthetic comprises particles having an average particle size of less than about 250 μm.

98. A method of treating post-surgical local pain of a subject using a sustained release dosage form, the method comprising:
administering once a short duration sustained release dosage form comprising a gel vehicle, which comprises a low molecular weight bioerodible, biocompatible lactic acid-based polymer, and a water-immiscible solvent in an amount effective to plasticize the polymer and form a gel therewith; an anesthetic comprising bupivacaine dissolved or dispersed in the gel vehicle; and a controllable efficacy ratio to achieve a release profile.

99. The method of claim 98 wherein the polymer comprises a copolymer of lactic acid and glycolic acid (PLGA).

100. The method of claim 99 wherein the copolymer has a monomer ratio of lactic acid to glycolic acid of approximately 50:50.

101. A method of preparing a sustained release dosage form, the method comprising:
preparing a short duration gel vehicle comprising a low molecular weight bioerodible, biocompatible polymer and a water-immiscible solvent in an amount effective to plasticize the polymer and form a gel therewith to create a polymer/solvent solution or gel;
equilibrating the polymer/solvent mixture until a clear homogeneous solution or gel is achieved;
dissolving or dispersing an anesthetic into the polymer/solvent solution or gel;
blending the anesthetic and the polymer/solvent solution or gel to form a sustained release dosage form; and
controlling an efficacy ratio to achieve a release profile.

102. The method of claim 101 wherein the efficacy ratio is between about 1 and 200.

103. The method of claim 101 wherein the polymer/solvent solution or gel is equilibrated at a temperature between room temperature and approximately 65° C.

104. The method of claim 101 wherein the anesthetic comprises bupivacaine.

105. The method of claim 101 wherein the anesthetic is selected from the group consisting of: bupivacaine, levo-bupivacaine, ropivacaine, levo-ropivacaine, tetracaine, etidocaine, levo-etidocaine, dextro-etidocaine, levo-etidocaine, dextro-etidocaine, levo-mepivacaine, and combinations thereof.

106. The method of claim 101 wherein the polymer comprises a lactic acid-based polymer.

107. The method of claim 106 wherein the polymer comprises a copolymer of lactic acid and glycolic acid (PLGA).

108. The method of claim 107 wherein the copolymer has a monomer ratio of lactic acid to glycolic acid of approximately 50:50.

109. The method of claim 107 wherein the polymer comprises poly(D,L-lactide-co-glycolide).

110. The method of claim 107 wherein the polymer comprises poly(L-lactide-co-glycolide).

111. The method of claim 101 comprising loading the dosage form with from about 0.1% to about 50% anesthetic by weight of the dosage form.

112. The method of claim 111 comprising loading the dosage form with from about 0.5% to about 40% anesthetic by weight of the dosage form.

113. The method of claim 112 comprising loading the dosage form with from about 1% to about 30% anesthetic by weight of the dosage form.

114. The method of claim 101 comprising providing a ratio of about 5:95 and about 90:10 between the polymer and the solvent.

115. The method of claim 114 comprising providing a ratio of about 20:80 and about 80:20 between the polymer and the solvent.

116. The method of claim 115 comprising providing a ratio of about 30:70 and about 75:25 between the polymer and the solvent.

117. The method of claim 101 wherein the solvent is selected from the group consisting of: an aromatic alcohol, lower alkyl esters of aryl acids, lower aralkyl esters of aryl acids; aryl ketones, aralkyl ketones, lower alkyl ketones, lower alkyl esters of citric acid, and combinations thereof.

118. The method of claim 107 wherein the PLGA comprises an ester end group.

119. The method of claim 107 wherein the PLGA comprises a carboxyl end group.

120. The method of claim 101 further comprising adding at least one of the following to the dosage form: an excipient, an emulsifying agent, a pore former, a solubility modulator for the anesthetic, and an osmotic agent.

121. The method of claim 101 wherein the anesthetic comprises particles having an average particle size of less than about 250 μm.

122. A kit for administration of a sustained delivery of an anesthetic to local pain of a subject comprising:
a short duration gel vehicle comprising a low molecular weight bioerodible, biocompatible polymer and a water-immiscible solvent, in an amount effective to plasticize the polymer and form a gel therewith;
an anesthetic dissolved or dispersed in the gel vehicle; and optionally, one or more of the following:
an excipient;
an emulsifying agent;
a pore former;
a solubility modulator for the anesthetic, optionally associated with the anesthetic; and
an osmotic agent;
wherein at the least anesthetic agent, optionally associated with the solubility modulator, is maintained separated from the solvent until the time of administration of the anesthetic to the subject.
